(19) United States
(12) Patent Application Publication
(10) Pub. No.: US 2009/0081292 A1
(43) Pub. Date: Mar. 26, 2009

(54) Oil-containing solid product and process for producing the same

(76) Inventors: Naoya Otomo, Kanagawa-ken (JP); Hirotsugu Kido, Kanagawa-ken (JP); Masahiko Ishitobi, Kanagawa-ken (JP); Kazuyuki Nishiyama, Kanagawa-ken (JP)

Correspondence Address: NIXON & VANDERHYE, PC, 901 NORTH GLEBE ROAD, 11TH FLOOR, ARLINGTON, VA, 22203, US

(21) Appl. No.: 11/919,865

(22) PCT Filed: Jun. 13, 2006
(86) PCT No.: PCT/JP2006/311821

§ 371 Date: Jul. 17, 2008

(30) Foreign Application Priority Data
Jun. 21, 2005 (JP) 2005-181205

(51) Int. Cl. A61K 9/18; C10L 5/00; A61K 47/34; A23D 9/007; B01J 13/00; C05F 11/00

(52) U.S. Cl. 424/484; 44/268; 71/64.09; 514/772.3; 516/108; 426/601

(57) ABSTRACT

An oil-containing solid in which liquid oil is contained in a large amount and from which oil seepage is slight; and a process for producing the same. The oil-containing solid is produced by impregnating a porous solid with a W/O emulsion. Further, use is made of the W/O emulsion having a water-soluble gellable substance incorporated in the water phase thereof. Consequently, the water-soluble gellable substance is converted to a gel in pores of the porous solid, thereby enhancing the liquid oil leakage preventing effect of the W/O emulsion.

### TECHNICAL FIELD

The present invention relates to an oil-containing solid product comprising a porous solid material and a W/O emulsion filled in pores of the porous solid material, and, more particularly, to an oil-containing solid product comprising a porous solid material and a large amount of a liquid oil contained and retained in pores of the porous solid material which undergoes a less leakage of the liquid oil therefrom, and a process for producing the oil-containing solid product.

### BACKGROUND ART

There are generally known techniques for imparting functions of a liquid oil to a solid material such as fertilizers and solid fuels by allowing the solid material to retain the liquid oil therein. These techniques aim at constraining and retaining the liquid oil in a specific region defined by the solid material to allow the solids to exhibit functions inherent to the liquid oil.

In the above oil-containing solid products, when using a porous solid material as the solid material, there tend to arise problems upon production of the oil-containing solid products such as “poor penetration of the liquid oil into pores of the porous solid material” and “difficulty in retaining a large amount of the liquid oil in the porous solid material owing to limited liquid oil absorption of the porous solid material”, as well as problems after production of the oil-containing solid products such as “leakage of the liquid oil from pores of the porous solid material owing to poor retention of the liquid oil in the pores of the porous solid material”.

To solve the above problems concerning leakage of the liquid oil from the oil-containing solid products, in the fields of foods and drugs, there has been proposed the method of encapsulating the liquid oil to prevent the liquid oil from being oozed and leaked out, thereby allowing components contained therein to be well retained in the solid products. However, in any other fields, the encapsulation of the liquid oil is unfavorable in some cases. Therefore, it tends to be very difficult to apply the “wrapping technique” such as encapsulation to all of the cases. Further, from the viewpoints of expenditures, costs, facilities, functions of the resultant products, it has been demanded to develop techniques other than the “wrapping technique” for retaining the liquid oil in the solid material.

For example, in the oil-containing solid products such as fertilizers, agricultural chemicals and aromatic agents, for the purpose of controlling an activity of effective components contained in the solid material or an activity-exhibiting time thereof, there has been proposed the method for producing an oil-containing solid product by directly impregnating a liquid oil or a perfume into a solid base material (Japanese Patent Application Laid-open (KOKAI) Nos. 2003-212708 and 10-127743 (1998)). Also, in the fields of diets for livestock and pisciculture, for the purpose of strengthening nutrition of the diets (increase in calorie and physiological activity), there has been proposed the method of producing an oil-containing solid product by adding and impregnating a liquid oil into solid diets (Japanese Patent Application Laid-open (KOKAI) No. 2004-236592).

In the above oil-containing solid products, in particular, in diets of a solid type used in the above diet fields, bleeding or leakage of oil therefrom is especially significant. The solid diets are obtained by blending and kneading mainly powders derived from animals or vegetables, gluten, starch, oils and fats, vitamins and minerals with each other and forming the resultant mixture into a solid product having a very high porosity. When a liquid oil is impregnated into the solid diets, the liquid oil tends to be leaked from voids in the solid diets, thereby causing such a problem that the diets tend to be deteriorated in nutritive value. Further, the leaked liquid oil tends to cause various problems such as poor quality of the diets owing to decrease in oil content upon distribution and storage thereof, deterioration in operability of diet feed devices and workability thereof and marine contamination after feed of the diets.

The existing techniques for producing an oil-containing solid product which does not depend upon the “wrapping technique” are generally classified into two methods in which one is. the method (1) of blending and kneading a liquid oil in solid raw materials upon production of the solid material and then molding and solidifying the obtained kneaded material, and the other is the method (2) of externally adding a liquid oil to the solid material as produced to obtain the aimed oil-containing solid product.

The technique of the method (1) relates, as described in Japanese Patent Application Laid-open (KOKAI) No. 8-109366 (1996), to such a technique and production method in which upon producing aromatic agents or detergents, a liquid oil (sometimes classified into perfumes in the case of the aromatic agents or detergents) is kneaded in a raw material, and then the resultant kneaded material is molded and the content of the liquid oil therein is adjusted, thereby controlling a releasability of the effective components from the resultant solid product.

However, in the technique of the method (1), when a large amount of the liquid oil is kneaded in the solid raw material to enhance a content of the liquid oil in the resultant solid product, there tends to arise the problem concerning strength of the kneaded material upon molding, so that the thus molded solid product tends to be deteriorated in shape retention property and, therefore, easily undergo breakage. In addition, an excessive amount of the oil component tends to be migrated through the solid material, thereby finally causing oil bleeding on the surface of the resultant solid product. Owing to these problems, it is not possible to knead a very large amount of the liquid oil in the solid material.

On the other hand, the technique of the method (2) relates, as described in Japanese Patent Application Laid-open (KOKAI) No. 9-201168 (1997), to such a technique for producing a high-oil content solid diet in which upon production of the solid diet, a diet base material having a small liquid oil content is first produced and solidified, and then a liquid oil is caused to absorb into pores of the obtained (porous) solid material, or retained in voids of the pores of the porous solid material by immersing the solid material in the liquid oil, etc.

However, in the technique of the method (2), when the solid material used is a porous solid material having voids, it may be very difficult to retain the liquid oil in the voids of the solid material, resulting in such a problem that “the liquid oil fails to be sufficiently retained in the solid material and tends to be leaked from the voids”.

### DISCLOSURE OF THE INVENTION

### Problem to be Solved by the Invention

An object of the present invention is to provide the technique for solving two problems including (1) the “difficulty in penetrating the liquid oil into pores” and (2) the “leakage of the liquid oil from the pores” which are encountered in the technical procedure of “impregnating the liquid oil into the solid material” among the techniques for producing the oil-containing porous solid product.

Also, another object of the present invention is to provide an oil-containing solid product capable of stably retaining the liquid oil in pores of the solid material and exhibiting a high oil content in the solid material, and a less oil leakage therefrom, as well as a process for producing the oil-containing solid product by using the above technique.

### Means for Solving Problem

As a result of the present inventors' earnest study for solving the above conventional problems, it has been found that by impregnating not a liquid oil itself but a W/O emulsion thereof into the porous solid material, the liquid oil can be stably retained at a high concentration in the solid material and can be prevented from being leaked therefrom. The present invention has been attained on the basis of the above finding.

More specifically, the liquid oil is dissolved in a surfactant and then mixed with a water phase to prepare a W/O emulsion thereof. Successively, the thus prepared W/O emulsion is impregnated into the porous solid material to thereby stably retain the W/O emulsion in pores of the solid material. Further, in the technique of the present invention, a gelatinizable substance is incorporated into the water phase to allow the substance to be gelled in voids of the solid material, thereby effectively preventing the liquid oil from being leaked from the solid material.

The present invention relates to the following aspects:

(1) An oil-containing solid product comprising a porous solid material and a W/O emulsion impregnated into pores of the porous solid material.

(2) An oil-containing solid product comprising a porous solid material and a W/O emulsion filled in pores of the porous solid material.

(3) An oil-containing solid product described in the above aspect (1) or (2), wherein a gel polymer is filled in the pores of the porous solid material.

(4) An oil-containing solid product described in any one of the above aspects (1) to (3), wherein a content of a water phase in the W/O emulsion is 0.01 to 50% by weight.

(5) An oil-containing solid product described in any one of the above aspects (1) to (4), wherein the water phase in the W/O emulsion contains a water-soluble gelatinizable substance.

(6) An oil-containing solid product described in any one of the above aspects (1) to (5), wherein the porous solid material is at least one solid material selected from the group consisting of foods, diets, solid fuels, aromatic agents, fertilizers and drugs.

(7) A process for producing an oil-containing solid product comprising the step of impregnating a W/O emulsion into a porous solid material.

(8) A process described in the above aspect (7), wherein the W/O emulsion contains a water-soluble polymer in a water phase thereof.

(9) A process described in the above aspect (8), wherein the water-soluble polymer is gelatinized in pores of the porous solid material.

### EFFECT OF THE INVENTION

In accordance with the present invention, it is possible to solve two problems including the “difficulty in penetrating a liquid oil into pores” and the “leakage of the liquid oil from the pores” which are encountered in the technique of impregnating the liquid oil into a porous solid material at a high concentration.

With the above technique, it is possible to stably retain the liquid oil in the porous solid material at a high concentration, thereby providing a solid product which is lessened in amount of the liquid oil leaked therefrom. Further, by preventing the liquid oil from being leaked from the solid material, it is possible to eliminate various problems caused due to leakage of the liquid oil and, therefore, efficiently exhibit functions of the liquid oil impregnated in the solid material.

Besides, in addition to the above effects, there is attained such a new merit that “a water-soluble substance usually incapable of being dissolved in the liquid oil can be retained in the oil-containing solid product”.

### PREFERRED EMBODIMENTS FOR CARRYING OUT THE INVENTION

In one aspect of the present invention, there is provided a solid-containing product obtained by impregnating a W/O emulsion into pores of a porous solid material.

First, penetration of the liquid oil into the porous material and leakage of the liquid oil therefrom as well as effects of the W/O emulsion are described.

<Penetration of Liquid Oil into Porous Solid Material>

The porous solid material is regarded as an aggregate of capillary tubes in view of its structure, and includes inside and outside voids. Penetration of the liquid oil into the porous solid material is generally divided into two steps including 1) a first step of contacting the liquid oil with the surface of the porous solid material and allowing the liquid oil to enter into pores of the porous solid material; and 2) a second step of allowing the liquid oil entering into the pores to migrate and move into deep portions of the porous solid material through the respective voids (pores) of the capillary tubes.

In the above two steps, it is considered that the following relationships between properties of the liquid oil and easiness of penetration of the liquid oil are established.

### Regarding the Step 1):

In general, the lower the interfacial tension between a solid and a liquid oil, the more easily the solid and the liquid oil are contacted with each other. More specifically, in the case where the liquid oil is penetrated into the porous solid material, the contact between the porous solid material and the liquid oil takes place in a more facilitated manner when the interfacial tension therebetween is lower. Therefore, in the step (1), the liquid oil preferably has a “low” interfacial tension to increase a concentration of the liquid oil in the porous solid material.

Regarding the step 2):

The step 2) is generally classified into two patterns according to the size of the pores. (1) When the size of the pores is large, a solution in the pores undergoes only a slight capillary phenomenon, so that movement of the liquid oil in the pores is caused mainly due to contact between the surface of the porous solid material and the liquid oil. Therefore, in the large pores, the interfacial tension of the liquid oil is preferably “low” from the viewpoint of good penetration thereof into the pores. On the other hand, (2) when the size of the pores is small (fine), a remarkable capillary phenomenon of the solution occurs. In this case, the higher the surface tension of the liquid, the stronger the force of causing the liquid to move though the capillary tubes becomes. The liquid having a high interfacial tension undergoes a force of reducing a surface area thereof in the capillary tubes. As a result, the force acts for moving the liquid along a wall surface of the capillary tubes. For example, water having a high surface tension (72 dyne/cm) owing to a strong intermolecular force thereof is easily penetrated into porous solid materials such as fibers and wood materials. This is because water has a high interfacial tension and, therefore, can be easily moved through the capillary tubes. Therefore, in fine portions of the pores, the liquid oil preferably has a “high” interfacial tension to facilitate movement of the liquid oil through an inside of the porous solid material and increase a concentration of the liquid oil in the porous solid material.

<Leakage of Liquid Oil from Porous Solid Material>

On the other hand, leakage of the liquid oil from the porous solid material tends to be caused in the following condition except for external factors such as application of load (pressure). That is, when the interfacial tension between the porous solid material and the liquid oil is low, the liquid oil failed to be retained on the surface of the porous solid material and within the pores thereof, thereby causing leakage of the liquid oil out of the pores. Also, when the viscosity of the liquid oil is lowered owing to factors such as temperature rise, the liquid oil is unable to be adsorbed into the pores, resulting in leakage of the liquid oil from the pores.

Thus, in order to stabilize the liquid oil in the porous solid material, the liquid oil preferably has a high interfacial tension.

### <Effectiveness of W/O Emulsion for Stabilization of Liquid Oil in Porous Solid Material>

In view of the above factors, in order to increase a concentration of the liquid oil in the porous solid material, it is required to suitably control the interfacial tension of the liquid oil under the three conditions including 1) entrance of the liquid oil from the surface of the porous solid material into the pores thereof; 2) movement of the liquid oil through the pores having a large size; and 3) movement of the liquid oil through the fine portions of the pores.

The present inventors have selectively used the “W/O emulsion” prepared by dispersing water in the liquid oil using a surfactant as a solution capable of “easily penetrating into the porous solid material and being stably continuously adsorbed into the pores of the porous solid material irrespective of change in external factors such as temperature change”.

The condition of penetrating and adsorbing the W/O emulsion into the porous solid material is explained as follows.

1) Condition in which the Liquid Oil is Contacted with the Surface of the Porous Solid Material and Enters into the Pores Thereof:

The liquid oil as a dispersing medium of the W/O emulsion is reduced in interfacial tension by dissolving a surfactant therein. More specifically, the ability of contacting the W/O emulsion with the surface of the porous solid material is enhanced by the action of the surfactant, so that the W/O emulsion can be more easily penetrated into the porous solid material as compared to the liquid oil solely.

2) Condition in which the Liquid Oil is Moved Through Pores Having a Large Size:

The W/O emulsion entering the pores undergoes only a slight capillary phenomenon when the porous solid material has a large pore size. As described above, under such a condition, the liquid oil preferably has a low interfacial tension for good movement thereof though the pores. Similarly to the condition 1), the interfacial tension of the liquid oil is lowered by dissolving the surfactant therein, so that the W/O emulsion can be easily penetrated into the porous solid material.

3) Condition in which the Liquid Oil is Moved Through Fine Portion of the Pores:

When the W/O emulsion further enters into the pores having a smaller pore size, the solution undergoes a more remarkable capillary phenomenon, and this capillary force acts as a driving force for allowing the liquid oil to move through the pores.

When the relationship of “(pore size of the porous solid material)≦(size of emulsified particles in the W/O emulsion)” is established, a water phase (emulsified droplets) as a dispersoid acts on the pores. As a result, the capillary force of water having a higher interfacial tension than that of the dispersing medium (liquid oil) is exhibited so that the W/O emulsion is more easily moved through the pores. Also, since water having a high interfacial tension is easily retained in the pores, the emulsion can be kept stably adsorbed into the pores.

More specifically, the liquid oil as a dispersing medium of the W/O emulsion is readily contacted with the inner and outer surfaces of the porous solid material and, therefore, can easily deliver water as a dispersoid into the pores. As described above, the emulsified droplets exhibit a good penetration into the pores and are easily retained in the pores owing to a high interfacial tension thereof, and further undergoes a less leakage of the liquid oil. The surfactant used in this technique decreases the interfacial tension between the liquid oil and water to form an emulsion, thereby acting for delivering water in a stable state into the pores. Further, the surfactant distributes a larger amount of the liquid oil into water retained in the pores, thereby acting for retaining the liquid oil therein.

As a result, it has been found that the liquid oil (emulsion) has a function of “readily penetrating into the porous solid material and being hardly leaked therefrom”, thereby achieving the present invention relating to the technical task of “increasing a concentration of the liquid oil in the porous solid material”.

In addition, the effect of preventing leakage of the liquid oil according to the present invention can be further enhanced by using the W/O emulsion prepared by incorporating a water-soluble gelatinizable substance into the water phase.

The water-soluble gelatinizable substance has the following effect. That is, after the emulsion is adsorbed into the pores of the solid material, the water-soluble gelatinizable substance contained in the emulsion is gelled, so that the liquid oil (emulsion) is more stably and firmly retained and adsorbed in the pores by the obtained gel, thereby preventing the liquid oil from being leaked therefrom.

In the followings, the porous solid material and the W/O emulsion used in the present invention are described in detail.

### (1) Porous Solid Material

The porous solid material used in the present invention may be those solid materials capable of filling the liquid oil in voids or pores inside thereof. For the standpoints of readily filling the liquid oil into the voids or pores and well retaining the liquid oil in the voids or pores, the size of the voids or pores in the porous solid material is usually not less than 0.001 μm and usually not more than 1000 μm, preferably not more than 500 μm and more preferably not more than 100 μm.

As long as the porous solid material used in the present invention is capable of retaining the liquid oil in the voids present in the solid material, the shape of the porous solid material is not particularly limited. As the porous solid material, there may be used those solid materials having voids such as irregularities and pores, on the surface and/or inside thereof.

The material of the porous solid material is not particularly limited. Examples of the material of the porous solid material include proteins, amino acids, lipids, carbohydrates and vitamins which are obtained from animals and plants as well as decomposed products and chemically modified products thereof, metals (minerals) and salts thereof, water, chemically and biologically synthesized polymers or the like.

Specific examples of the porous solid material include diets for livestock and pisciculture (solid diets), pet foods, foods such as cookies and sponge cakes, chemical fertilizers or organic fertilizers, solid aromatic agents, solid deodorizers, solid deodorants, solid detergents, solid fuels, cosmetics, solid bath agents, fiber masses, felts, wood materials, straws, soils, glass or resin hollow materials or the like.

The solid piscicultural diets may be produced, for example, by mixing main raw materials such as fish meals, soybean oil meals, corn gluten meals, krill meals, starches and rice bran, if required, with vitamins, minerals, calcium carbonate, calcium phosphate, etc., and then pressing and extruding the resultant mixture using an extruder (of a single-screw or twin-screw type).

The thus produced solid piscicultural diets are in the form of a porous solid material having voids and usually have a cylindrical shape. The size of the solid piscicultural diets varies depending upon kind and degree of growth of fishes to be cultivated, and can be optionally selected from small ones having a diameter of 2 to 4 mm through large ones having a diameter of 20 to 25 mm.

### (2) W/O Emulsion

The W/O emulsion used in the present invention may be produced from an oil component, a surfactant and an aqueous solution (water phase).

The amount of the W/O emulsion filled is usually not less than 0.01% by weight and also usually not more than 80% by weight based on the weight of the porous solid material.

In addition, the W/O emulsion used in the present invention has such a merit that a substance usually undissolvable in the oil component is dissolved in the water phase and imparted to the liquid oil and solid material. More specifically, both of an oil-soluble substance and a water-soluble substance can be used in the emulsion without any particular limitations.

Therefore, according to the aimed objects, antioxidants, antiseptics, pigments, sugars, salts, seasonings, dairy products, etc., may be appropriately added to the aqueous solution (water phase) or the oil component.

### (a) Oil Component

As the oil component contained in the W/O emulsion produced according to the present invention, there may be used known oil components utilized in the fields of foods, diets, cosmetics, drugs and industries without any particular limitations. Examples of the oil component include animal and vegetable oils and fats, fatty acids and esters thereof with alcohols, hydrocarbons, saturated or unsaturated higher alcohols, waxes, essential oils, oleoresins and resinoids, perfumes, and enzymatically treated (hydrolysis, transesterification, etc.) or chemically treated (transesterification, hydrogenation, etc.) products thereof.

Specific examples of the animal and vegetable oils and fats include fish oil, beef tallow, lard, milk fat, horse oil, snake oil, egg oil, egg yolk oil, soybean oil, maize oil, cotton seed oil, rape seed oil, sesame oil, perilla oil, rice oil, sunflower oil, arachis oil, olive oil, palm oil, palm kernel oil, rice embryo oil, wheat embryo oil, unpolished rice embryo oil, adlay oil, garlic oil, jojoba oil, macadamia nut oil, avocado oil, eucalyptus oil, evening primrose oil, turtle oil, mink oil, flower oils, tsubaki oil, coconut oil, castor oil, linseed oil, cacao oil, medium-chain fatty acid triglycerides, and processed oils and fats obtained by subjecting these oils and fats to hydrogenation or transesterification.

Specific examples of the fatty acids and esters thereof with alcohols include myristic acid, palmitic acid, isopalmitic acid, stearic acid, oleic acid, linoleic acid, linolenic acid, ricinoleic acid, 12-hydroxystearic acid, 10-hydroxystearic acid, behenic acid, hexadecatrienoic acid, octadecatrienoic acid, eicosatetraenoic acid, docosatetraenoic acid, eicosapentaenoic acid, docosapentaenoic acid, docosahexaenoic acid and tetrahexaenoic acid, as well as geometrical isomers of these acids and esters of these acids with alcohols.

Specific examples of the hydrocarbons include light liquid paraffin, heavy liquid paraffin, liquid isoparaffin, light liquid isoparaffin, ceresin, paraffin, microcrystalline waxes, vaseline, squalane, squalene, etc.

Specific examples of the saturated or unsaturated higher alcohols include alcohols having 8 to 44 carbon atoms such as lauryl alcohol, myristyl alcohol, cetanol, stearyl alcohol, oleyl alcohol, isostearyl alcohol and 2-octyl dodecanol octacosanol.

Specific examples of the waxes include jojoba oil, rice wax, propolis, beeswax, bleached beeswax, candelilla wax, carnauba wax, Japan wax, spermaceti, ceresin, etc.

Specific examples of the essential oils include ambrette seed oil, mustard oil, saffron oil, citronella oil, vetiver oil, valerian oil, segebush oil, chamomil oil, camphor oil, sassafras oil, Ho leaf or wood oil, rosewood oil, clary sage oil, thyme oil, basil oil, carnation oil, cedar wood oil, cypress oil, white cedar oil, clove oil, turpentine oil, pine oil, etc.

Specific examples of the oleoresins or resinoids include pepper, cardamon, ginger, parsley, coriander, caraway, pimenta, vanilla, celery, clove, nutmeg, paprike, orris resinoids, mastiche, etc.

Specific examples of the perfumes include orange oil, lemon grass oil, tarragon oil, avocado oil, bay leaf oil, cassia oil, cinnamon oil, pepper oil, calamus oil, sage oil, mint oil, peppermint oil, spearmint oil, patchouli oil, rosemary oil, lavandula oil(?), lavender oil, curcuma oil, cardamon oil, ginger oil, angelica oil, anise oil, fennel oil, parsley oil, celery oil, galbanum oil, cumin oil, coriander oil, dill oil, carrot oil, caraway oil, winter green oil, nutmeg oil, rose oil, cypress oil, sandalwood oil, allspice oil, grapefruit oil, neroli oil, lemon oil, lime oil, bergamot oil, mandarin oil, onion oil, garlic oil, bitter almond oil, geranium oil, mimosa oil, jasmine oil, fragrant olive oil, star anise oil, cananga oil, ilang-ilang oil, eugenol, ethyl caprylate, geraniol, menthol, citral, citronellol, borneol, etc.

The above respective components may be used alone or in combination of any two or more thereof at the same time.

### <Hardened Oils and Fats>

The oil component may also contain hardened oils and fats for the purpose of attaining a higher effect of preventing leakage of the liquid oil. As the hardened oils and fats, there may be used hydrogenated products of animal and vegetable oils and fats or those oils and fats obtained by separating a high-boiling fraction from the animal and vegetable oils and fats. Specific examples of the hardened oils and fats include hardened coconut oil, hardened palm kernel oil, hardened herring oil, hardened cod liver oil, hardened beef tallow, hardened palm oil, hardened cotton seed oil, hardened olive oil, hardened arachis oil, hardened soybean oil, hardened linseed oil, hardened castor oil, etc. These hardened oils may be used alone or in combination of any two or more thereof.

The amount of the oil component filled is usually not less than 0.01% by weight and also usually not more than 80% by weight based on the weight of the porous solid material.

The oil component may also optionally contain an oil-soluble substance such as antioxidant according to the requirements. Examples of the oil-soluble antioxidant include oil-soluble rosemary extracts, tea extracts, catechin, epicatechin, epigallocatechin, catechin gallate, epigallocatechin gallate, vitamin E (α, β, γ, δ-tocopherol), mixed tocopherol, vitamin C fatty acid esters, etc.

### (b) Surfactant

The surfactant used in the present invention is preferably capable of forming such a W/O emulsion in which a water phase is stably dispersed in an oil phase. As the surfactant, there may be used any known surfactants generally used in the application fields such as foods, diets, cosmetics, drugs and industries without any particular limitations.

The surfactants are classified into ionic surfactants, nonionic surfactants, amphoteric surfactants, etc., from the standpoint of chemical properties thereof. In the present invention, any of these surfactants may be used.

Further, the surfactants are classified into industrial surfactants, emulsifiers for foods, natural surfactants, etc., from the standpoint of applications thereof. In the present invention, although any of these surfactants may be used, from the standpoints of safety for environments and organisms as well as applications to beverages and foods, diets or cosmetics, among these surfactants, preferred are emulsifiers for foods and natural surfactants. In addition, among the emulsifiers for foods, from the viewpoints of a good availability, a capability of selecting broader HLB values and broader kinds of fatty acids and no particular limitations to domestic use, more preferred are sucrose fatty acid esters or polyglycerol fatty acid esters. These surfactants may be used alone or in combination of any two or more thereof.

### <Emulsifiers for Foods>

Examples of the emulsifiers for foods include sucrose fatty acid esters, polyglycerol fatty acid esters, glycerol fatty esters, glycerol acetic acid fatty acid esters, glycerol lactic acid fatty acid esters, glycerol succinic acid fatty acid esters, glycerol citric acid fatty acid esters, glycerol diacetyl tartaric acid fatty acid esters, polyglycerol condensed ricinoleic acid fatty acid esters, sucrose acetic acid isobutyric acid fatty acid esters, sorbitan fatty acid esters, propylene glycol fatty acid esters, lecithin, calcium stearoyl lactate (CSL), oxyethylene higher fatty alcohols, polyoxyethylene higher fatty alcohols, sodium oleate, morpholine fatty acid salts, etc.

The fatty acids as a constituent of the above fatty acid ester compounds are usually in the form of a fatty acid or a hydroxy-fatty acid having 8 to 24 carbon atoms. The hydrocarbon group or hydroxy-hydrocarbon group contained in the fatty acids may be either linear or branched, and either saturated or unsaturated. Specific examples of the fatty acids include caprylic acid, capric acid, lauric acid, myristic acid, palmitic acid, stearic acid, arachic acid, behenic acid, tetradecenoic acid, hexadecenoic acid, octadecenoic acid, octadecadienoic acid, eicosenoic acid, eicosatetraenoic acid, docosenoic acids such as erucic acid, octadecatrienoic acid, isopalmitic acid, isostearic acid, ricinolic acid, 12-hydroxystearic acid, etc. Among these fatty acids, from the viewpoints of an excellent flexibility upon use and a good availability, preferred are those fatty acids having 12 to 24 carbon atoms, and from the viewpoint of facilitating hardening of the liquid oil (high boiling point), more preferred are those fatty acids having 18 to 22 carbon atoms. These fatty acids may be used in combination of any two or more thereof according to the aimed applications.

### <Sucrose Fatty Acid Esters>

The sucrose fatty acid esters usually contain those esters having different esterification degrees from each other such as mono-, di-, tri-, tetra- and higher esters, and may be frequently used in the form of a mixture of these esters. The sucrose fatty acid esters used in the present invention preferably exhibit a higher affinity to the oil component upon forming the W/O emulsion. Such sucrose fatty acid esters are those having a high average esterification degree and a HLB (hydrophile-lipophile balance) value of preferably 1 to 8 and more preferably 1 to 6.

Examples of commercially available products of the sucrose fatty acid esters include “S-170” (sucrose stearic acid ester; HBL: 1; produced by Mitsubishi-Kagaku Foods Corporation), “ER-290” (sucrose erucic acid ester; HLB: 2; produced by Mitsubishi-Kagaku Foods Corporation), etc.

### <Polyglycerol Fatty Acid Esters>

The polyglycerol fatty acid esters are usually produced by reacting polyglycerol with a fatty acid. As the polyglycerol, there are generally used those having an average polymerization degree of 2 to 16. These polyglycerols may be obtained in the form of a linear, branched or cyclic polymer or a mixture thereof according to the type of synthesis reaction thereof. Also, by controlling an esterification degree of the fatty acid, it is possible to synthesize polyglycerol fatty acid esters having various HLB values. The polyglycerol fatty acid esters used in the present invention have an average polymerization degree of polyglycerol of 2 to 16 and preferably 4 to 12. In addition, the polyglycerol fatty acid esters preferably exhibit a higher affinity to the oil component upon forming the W/O emulsion. The suitable polyglycerol fatty acid esters are those having a high average esterification degree and a HLB value of preferably 1 to 8 and more preferably 1 to 6.

Examples of commercially available product of the polyglycerol fatty acid esters include “ER-60D” (decaglycerol erucic acid ester; HLB: 5; produced by Mitsubishi-Kagaku Foods Corporation), “B-100D” (decaglycerol behenic acid ester; HLB: 3; produced by Mitsubishi-Kagaku Foods Corporation), etc.

### <Natural Surfactants>

Examples of the natural surfactants include vegetable lecithin, yolk lecithin, fractionated lecithin, enzyme-treated lecithin, saponin, quillaja saponin, soybean saponin, sphingolipid, vegetable sterol, animal sterol, bile powder, tomato glycolipid, yucca foam extracts, etc.

### <Ionic Surfactants>

Examples of the anionic surfactants include base materials for soaps, fatty acid salts, sulfated oils, salts of higher alcohol sulfuric acid esters, salts of higher alkyl ether sulfuric acid esters, salts of higher fatty acid ester sulfuric acid esters, salts of secondary alcohol sulfuric acid esters, salts of higher fatty acid alkylolamide sulfuric acid esters, higher fatty amide sulfonic acid salts, higher fatty acid ester sulfonic acid salts, alkylbenznesulfonic acid salts, sulfosuccinic acid esters, etc.

The cationic surfactants are generally in the form of a compound obtained by replacing a hydrogen atom of an ammonium salt with an alkyl group. Examples of the cationic surfactants include mono-, di- and trialkyl ammonium salts, alkyl trimethyl ammonium salts, benzalconium chloride, quaternary ammonium salts such as pyridinium salts, polyethyleneoxide (POE)-alkyl amines, polyamine fatty acid derivatives, amyl alcohol fatty acid derivatives, organic modified clay minerals, etc.

### <Nonionic Surfactants>

Examples of the nonionic surfactants include “ether-type nonionic surfactants” containing alkyl phenols, higher fatty acids, alkyl amines, alkyl amides, polypropylene glycol, etc., and “polyhydric alcohol-type nonionic surfactants” containing, as a hydrophilic group, a polyhydric alcohol such as glycerol, sorbitol and sugar. The latter type nonionic surfactants are the same as described above in the paragraph “emulsifiers for foods”.

### <Amphoteric Surfactants>

Examples of the amphoteric surfactants include those containing a carboxylic acid salt, a sulfuric acid ester salt, a sulfonic acid salt, a phosphoric acid salt, etc., as an anion portion thereof. Among the amphoteric surfactants of a carboxylic acid salt type, preferred are betaine-based surfactants containing a quaternary ammonium salt as a cation portion thereof and a carboxylic acid salt as a cation portion thereof such as alkyl betaines, amide betaines and sulfo-betaines, imidazoline-based surfactants containing an imidazole ring in a cation portion thereof, amino acid-based surfactants containing an amino acid in a cation portion thereof, or the like.

The amount of these surfactants added is usually not less than 0.01% by weight and preferably not less than 0.1% by weight, and also usually less than 20% by weight and preferably less than 10% by weight based on the weight of the W/O emulsion.

### (c) Aqueous Solution (Water Phase):

In the present invention, the aqueous solution (water phase) cooperates with the oil component and the surfactant to form the W/O emulsion, and determines a water phase content in the W/O emulsion. The water phase content in the W/O emulsion is usually not less than 0.01% by weight, preferably not less than 0.05% by weight and more preferably not less than 0.1% by weight, and also usually not more than 50% by weight, preferably not more than 20% by weight and more preferably not more than 10% by weight.

The aqueous solution may comprise water solely, and may further contain other optional water-soluble substances, if required. As described above, another feature of the present invention resides in that the W/O emulsion containing a water-soluble substance usually undissolved in the oil component which is dissolved in the water phase is impregnated into the porous solid material, thereby enabling the water-soluble substance undissolved in the oil component to be incorporated into the liquid oil and the solid material according to the aimed applications and objects.

The water-soluble substance is not particularly limited, and any known water-soluble substances may be optionally used. As the water-soluble substances, in view of applications thereof, there may be optionally selected, for example, antioxidants, sweeteners, colorants, emulsifiers, preservatives, seasonings, perfumes, condiments, thickening stabilizers, bleaching agents, etc. These water-soluble substances may be used alone or in combination of any two or more thereof. Examples of the antioxidants include water-soluble natural extracts such as vitamin C and water-soluble rosemary extracts.

Since the W/O emulsion used in the present invention contains the oil component, it is expected that the oils and fats suffer from deterioration during the respective processes such as production, distribution and storage. In particular, in the W/O emulsion, it is expected that the oils and fats are deteriorated by adverse influence of oxygen dissolved in the water phase, etc. In order to prevent the oils and fats from being deteriorated, antioxidants may be added to the water phase, if required.

### (d) Method for Producing W/O Emulsion:

The W/O emulsion used in the present invention may be produced by using any known methods without particular limitations. First, the aimed surfactant is dissolved under heating in the liquid oil, and then the aqueous solution containing the gelatinizable substance is dispersed in the liquid oil. The emulsifying and dispersing method is not particularly limited as long as emulsified droplets are suitably formed by the method. However, in order to allow the emulsified droplets to be more completely dispersed in the resultant emulsion, there is preferably used such a production method in which the above respective components are uniformly dispersed and mixed using a propeller mixer, a cutter mixer, an agitation emulsifying device, a high-pressure homogenizer, a colloid mill, supersonic emulsification, membrane emulsification, a valve homogenizer, etc.

The particle size of the emulsified droplets formed upon producing the W/O emulsion is usually not less than 0.01 μm, preferably not less than 0.1 μm and more preferably not less than 1 μm, and also usually less than 500 μm, preferably less than 200 μm and more preferably less than 100 μm, though not limited thereto.

The resultant W/O emulsion may be directly impregnated as the liquid oil into the porous solid material. Alternatively, the W/O emulsion may be further mixed with other oil components, and the resultant mixture may be impregnated as the liquid oil into the porous solid material.

(3) Water-Soluble Gelatinizable Substance:

When adding the water-soluble gelatinizable substance to the water phase contained in the W/O emulsion used in the present invention, the emulsified droplets are gelled, thereby enabling the W/O emulsion to be more stably adsorbed into the pores. As the water-soluble gelatinizable substance, there may be used known water-soluble gelatinizable components utilized in the application fields such as foods, diets, drugs, cosmetics and industries without any particular limitations.

Specific examples of the water-soluble gelatinizable substance include polysaccharides, proteins, polyethylene, polyesters, polyamides, polyvinyl alcohol, polyvinyl aldehyde, acrylic polymers, polytetrafluoroethylene, polyacrylic acids, polyethylene glycol, polyvinyl alcohol-Cu²⁻, polyacrylic acid-Fe³⁺, polyvinylbenzyltrimethyl ammonium and derivatives thereof. Among these substances, from the standpoints of safety for environments and organisms as well as applications to beverages and foods, diets or cosmetics, preferred are polysaccharides and proteins. In addition, these water-soluble gelatinizable substances may be used alone or in combination of any two or more thereof.

Examples of the polysaccharides include those known as food additives such as starches, agars, carboxymethyl cellulose, methyl cellulose, hydroxypropyl cellulose, konjak-mannan, alginic acid, hyaluronic acid, guar gum, xanthan gum, carageenan, locust bean gum, gum arabic, tragacanth gum, tamarind gum, pectin, pullulan, curdlan, gellan gum and agarose. Among these polysaccharides, preferred are alginic acid, guar gum, xanthan gum, carageenan and locust bean gum from the viewpoint of good effects thereof.

Examples of the proteins include whey proteins, casein milk proteins, soybean proteins, wheat proteins, livestock proteins, fish proteins, gelatin, collagen, egg proteins, albumen proteins, serum proteins, fibrin, elastin, keratin, etc.

The concentration of the water-soluble gelatinizable substance added is usually not less than 0.1% by weight and also usually not more than 20% by weight and preferably not more than 10% by weight based on the weight of the water phase.

### (4) Gelatinization (Gel-Forming Reaction):

The gel-forming reaction of the water-soluble gelatinizable substance is basically a “reaction for forming a crosslinking structure” between molecules of the water-soluble gelatinizable substance which is induced by heat, light, pressure, electricity, plasma, radiation, catalyst, change in pH and ion strength, radicals, polyvalent cations, hydrophobic substance, etc. When the water-soluble gelatinizable substance contained in the aqueous solution dispersed in the W/O emulsion existing in voids within the pores of the porous solid material is gelled (subjected to the gel-forming reaction), the liquid oil (oil component) existing in the voids can be prevented from being leaked therefrom. As a result, it becomes possible to enhance an ability for retaining the liquid oil in the porous solid material.

Specific examples of the gel-forming reaction include crosslinking or polymerization reactions between molecules due to covalent bond, hydrogen bond, ionic bond, coordinate bond, hydrophobic bond Coulomb force or Van der Waals force between molecules or between functional groups within the molecule, reactions in which after forming physical entanglement of molecular chains and double helix between polymer chains, the resultant product is coagulated to form a crosslinking region, reactions in which a three-dimensional network structure is constructed due to modification of proteins induced by change in composition of solvents such as pH and ion strength, high pressure, cooling, heating or addition of modifying agents as well as association between the modified proteins, or random steric interaction between molecules, or the like.

Actually, a gelation assistant capable of inducing gelation of the water-soluble gelatinizable substance may be added to the porous solid material or the W/O emulsion. As the gelatinization assistant, various different kinds of compounds may be used depending upon the mechanism of gelation of the water-soluble gelatinizable substance.

Examples of the combination of the water-soluble gelatinizable substance and the gelation assistant include (1) combination of sodium alginate and a polyvalent cation such as Ca ion, (2) combination of polysaccharide hydrocolloids such as combination of xanthan gum and locust bean gum, (3) combination of a protein and an acid or alkali compound, or the like.

The gelation reaction mechanism induced by the gelation assistant is more specifically explained below according to the above combinations. That is, there may be exemplified the following reaction mechanisms, i.e., (1) a reaction mechanism in which a carboxyl group on a sugar chain is associated with a helix sugar chain through the polyvalent cation, and the resultant associated product forms a three-dimensional network, resulting in gelation thereof; (2) a reaction mechanism in which the second polymer is incorporated into a network of the first polymer so that both the polymers are sterically entangled with each other and gelled; (3) a reaction mechanism in which isoelectric point precipitation is caused by change in pH of ambient environment of proteins, thereby inducing the gelation, or the change in pH causes structural change of the proteins, resulting in solidification (modification) and gelation thereof; or the like.

The method of adding the gelation assistant is not particularly limited as long as the gelatinizable substance contained in the W/O emulsion can be contacted with the gelation assistant in the solid material. When the gelation assistant is previously added to a base material of the porous solid material, it is possible to induce gelation of the gelatinizable substance contained in the W/O emulsion impregnated into the porous solid material. As the method of previously adding the gelation assistant to the base material of the porous solid material, there may be used, for example, the method of kneading the gelation assistant in a raw material of the porous solid material, the method of adding the gelation assistant to the porous solid material before immersing the porous solid material in the liquid oil, or the like.

The concentration of the gelation assistant added varies depending upon the kind thereof, and is usually not less than 0.01% by weight and preferably not less than 0.1% by weight and also usually less than 10% by weight and preferably less than 1% by weight based on the weight of the porous solid material.

(5) Method of Impregnating W/O Emulsion into Porous Solid Material:

The method of impregnating the W/O emulsion into voids of the porous solid material is not particularly limited as long as the W/O emulsion as a liquid substance can be suitably absorbed into the porous solid material. In view of simplicity, after immersing the porous solid material in the W/O emulsion solution, the porous solid material is allowed to stand under normal pressures to penetrate the liquid oil into voids thereof. In an alternative method using a special equipment, the W/O emulsion may be impregnated into voids of the porous solid material by conducting pressurization, reduction in pressure, spraying and injection using a pressing machine, a pressure-reducing device, a sprayer, an injector, etc.

### (6) Method of Evaluating Oil-Containing Solid Product:

The oil-containing solid product of the present invention can be evaluated by the following oil leakage testing method.

In the oil leakage test, the oil-containing solid product is placed on 10 circular filter papers cut into a diameter of 5 cm (“No 5A” produced by ADVANTEC CORP.) and allowed to stand at 45° C. under normal pressures for 24 hr to cause the liquid oil in the solid product to be leaked out therefrom. Meanwhile, the effect of preventing leakage of the liquid oil is evaluated by the following method.

### <Evaluation for Effect of Preventing Leakage of Oil>

The weights of (A) the porous solid material, (B) the liquid oil- or W/O emulsion-impregnated porous solid material (oil-containing solid product) and (C) the oil-containing solid product after being allowed to stand for 24 hr, are respectively measured to calculate an oil content (%) and an oil leakage rate (%) of the oil-containing solid product according to the following formulae.

Oil content (%)=100×[(weight (B) of liquid oil-impregnated porous solid material)−(weight (A) of porous solid material before impregnating liquid oil thereinto)]/(weight (B) of liquid oil-impregnated porous solid material) (Formula 1)

Oil leakage rate (%)=100×[(weight (B) of liquid oil-impregnated porous solid material)−(weight (C) of oil-containing product after undergoing oil leakage)]/[(weight (B) of liquid oil-impregnated porous solid material)−(weight (A) of porous solid material before impregnating liquid oil thereinto)] (Formula 2)

That is, in the oil-containing solid product, the oil content calculated from the formula 1 is preferably higher, and the oil leakage rate calculated from the formula 2 is preferably lower. Therefore, the oil-containing solid product having a higher oil content and a lower oil leakage rate is regarded as being capable of “retaining a larger amount of the liquid oil and preventing the liquid oil from being leaked out therefrom”.

The oil content in the oil-containing solid product of the present invention is preferably not less than 10%, more preferably not less than 15% and still more preferably not less than 20%.

The oil leakage rate of the oil-containing solid product of the present invention is preferably not more than 55%, more preferably not more than 30% and still more preferably not more than 20%.

### EXAMPLES

The effects of the present invention are described in more detail by the following Examples and Comparative Examples. However, the following Examples are not intended to limit the scope of the present invention.

### (1) Studies on Soybean Oil-Impregnated Solid Piscicultural Diets:

As an example showing the “effect of preventing leakage of the liquid oil” according to the present invention in the “solid diet” field where the oil leakage leads to especially significant problem, the results of studies on the effect of preventing leakage of the liquid oil from solid piscicultural diets are described below.

### Production Example 1

### Production of Solid Piscicultural Diets

After fully mixing the below-mentioned raw materials using a mixer, the resultant mixture was supplied with water, pressurized, molded and dried (up to water content of 10 to 15%) using a twin-screw extruder under extrusion conditions including a barrel temperature of 80 to 120° C. and an outlet pressure of 4 to 8 bars, thereby obtaining porous solid piscicultural diets.

Fish meal: 43% by weight

Soybean meal: 30% by weight

Starch and wheat flour: 12% by weight

Others (animal oils and fats, vitamins and minerals): 3% by weight

As a result, it was confirmed that the thus obtained solid piscicultural diets was of a size usable as piscicultural diets in broadest applications, i.e., had a weight of 1.3±0.1 (g) (1.2 to 1.5 (g)), a diameter of 12.3 to 13.9 mm and a height of 13.1±0.1 mm.

### Production Example 2

### Production of W/O Emulsion

Five parts by weight of decaglycerol erucic acid ester as a polyglycerol fatty acid ester (“ER-60D” produced by Mitsubishi-Kagaku Foods Corporation) was added to 94 parts by weight of a soybean oil (ointment base material; Japan Pharmaceutical Codex; produced by KOZAKAI PHARMACEUTICAL CO., LTD), and then the obtained mixture was heated to 75° C. and uniformly dissolved. The resultant solution was mixed with 1-part by weight of a 1 wt % aqueous solution of sodium alginate (“I-3G” produced by Kimica Corporation) and stirred under heating to prepare a uniform W/O emulsion. Three parts by weight of the thus obtained W/O emulsion was added and diluted in 22 parts by weight of a soybean oil previously heated to 60° C. to prepare a liquid oil containing the W/O emulsion which was subsequently used for immersing the solid piscicultural diets therein.

### Example 1

### Production of Solid Piscicultural Diets Impregnated with W/O Emulsion

An optional weight of the porous solid piscicultural diets obtained in Production Example 1 were weighed in a beaker, and then the liquid oil containing the W/O emulsion produced in Production Example 2 was filled in the beaker such that a whole part of the solid piscicultural diets was immersed in the liquid oil. The contents of the beaker were held at 60° C. under a reduced pressure of −0.085 to −0.095 MPa for 1 min, and then returned again to normal pressures to impregnate the W/O emulsion into the pores of the solid piscicultural diets. Only the thus impregnated solid piscicultural diets were recovered from the beaker, and the liquid oil attached onto the surface of the solid diets was lightly wiped off, thereby obtaining an oil-containing solid product.

In the oil leakage test, the oil-containing solid product was placed on 10 circular filter papers cut into a diameter of 5 cm (“No 5A” produced by ADVANTEC CORP.) and allowed to stand at 45° C. under normal pressures for 24 hr to cause the liquid oil in the solid product to be leaked out therefrom. Meanwhile, the effect of preventing leakage of the liquid oil was evaluated by the following method.

<Evaluation for Effect of Preventing Leakage of Oil from Oil-Containing Solid Piscicultural Diets>

The weights of (A) the solid piscicultural diets used, (B) the liquid oil- or W/O emulsion-impregnated solid piscicultural diets and (C) the solid piscicultural diets after being allowed to stand for 24 hr, were respectively measured to calculate an oil content (%) and an oil leakage rate (%) of the solid piscicultural diets according to the following formulae (3) and (4) similarly to the above formulae (1) and (2).

Oil content (%)=100×[(weight (B) of liquid oil-impregnated solid piscicultural diets)−(weight (A) of solid piscicultural diets)]/(weight (B) of liquid oil-impregnated solid piscicultural diets) (Formula 3)

Oil leakage rate (%)=100×[(weight (B) of liquid oil-impregnated solid piscicultural diets)−(weight (C) of solid piscicultural diets after undergoing oil leakage)]/[(weight (B) of liquid oil-impregnated solid piscicultural diets)−(weight (A) of solid piscicultural diets)] (Formula 4)

That is, in the oil-containing solid piscicultural diets, the oil content calculated from the formula 3 is preferably higher, and the oil leakage rate calculated from the formula 4 is preferably lower. Therefore, the oil-containing solid piscicultural diets having a higher oil content and a lower oil leakage rate was regarded as being capable of “retaining a larger amount of the liquid oil and preventing the liquid oil from being leaked out therefrom”.

### Comparative Example 1

The same procedure as defined in Example 1 was conducted except that only the soybean oil was impregnated into the piscicultural diets, thereby obtaining an oil-containing solid product. The thus obtained oil-containing solid product was subjected to the same oil leakage test as defined in Example 1.

The results of Example 1 and Comparative Example 1 are shown in Table 1.

TABLE 1

| | Additives in liquid oil | | | |
|---|---|---|---|---|
| | (soybean oil) | | Content of | |
| | | W/O emulsion | liquid oil | |
| | | (containing | before oil | Oil |
| | | gelatinizable | leakage | leakage |
| | ER-60D | substance) | test (%) | rate (%) |
| Example 1 | + | + | 23.9 ± 0.5 | 53.4 ± 2.2* |
| Comparative | − | − | 24.8 ± 0.2 | 61.5 ± 1.5 |
| Example 1 | | | | |

Note
*As a result of conducting the significant test (student t test) relative to Comparative Example 1, since p was less than 0.05 (p < 0.05), a significant difference (n = 3) was recognized.

As apparently recognized from Table 1, the W/O emulsion-containing solid piscicultural diets produced according to the present invention (Example 1) exhibited a less leakage of the liquid oil impregnated in the solid diets as compared to that of Comparative Example 1.

In the above system, it was considered that when the W/O emulsion was impregnated into the solid piscicultural diets, the aqueous sodium alginate solution contained in the W/O emulsion was contacted with a calcium ion derived from calcium carbonate or calcium phosphate as an additive of the solid diets, thereby inducing gelation of the alginate. As a result, it was suggested that the emulsified particles in the W/O emulsion were gelled, and voids in the solid piscicultural diets (porous solid material) were closed and filled with the resultant gel so as to prevent leakage of the liquid oil from the voids.

(2) Studies on Solid Piscicultural Diets Impregnated with Soybean Oil and Hardened Oils and Fats:

The effect of preventing leakage of the liquid oil according to the present invention in the case of previously adding the emulsifier and the hardened oils and fats to the liquid oil was examined as follows.

### Example 2

### Production of Solid Piscicultural Diets Impregnated with W/O Emulsion Containing Hardened Oils and Fats

After adding 8.3 parts by weight of decaglycerol behenic acid ester as a polyglycerol fatty acid ester (“B-100D” produced by Mitsubishi-Kagaku Foods Corporation) and 25 parts by weight of a 25 wt % hardened oil (melting point: about 60° C. (broad); “Z-4110” produced by Fuji Oil Co., Ltd.) to 66.7 parts by weight of a soybean oil, the obtained mixture was heated to 75° C. and uniformly dissolved. The resultant solution was mixed with 1 part by weight of a 1 wt % aqueous solution of sodium alginate (“I-3G” produced by Kimica Corporation) and stirred under heating to prepare a uniform W/O emulsion. Three parts by weight of the thus obtained W/O emulsion was mixed and diluted in 22 parts by weight of a soybean oil previously heated to 60° C. to prepare a liquid oil containing the W/O emulsion which was subsequently used for immersing the porous solid material therein. The resultant W/O emulsion-containing liquid oil was impregnated into the solid piscicultural diets by the same method as defined in Example 1, thereby obtaining an oil-containing solid product. The thus obtained oil-containing solid product was subjected to oil leakage test by the same method as defined in Example 1.

### Comparative Example 2

The same procedure as defined in Example 1 was conducted except that only the soybean oil was impregnated into the piscicultural diets, thereby obtaining an oil-containing solid product. The thus obtained oil-containing solid product was subjected to the same oil leakage test as defined in Example 1.

Comparative Example 3

A liquid oil prepared by mixing 8.3 parts by weight of decaglycerol behenic acid ester as a polyglycerol fatty acid ester (“B-100D” produced by Mitsubishi-Kagaku Foods Corporation) and 25 parts by weight of a hardened oil (melting point: about 60° C. (broad); “Z-4110” produced by Fuji Oil Co., Ltd.) with 66.7 parts by weight of a soybean oil, was impregnated into the solid piscicultural diets, and the resultant oil-containing solid product was subjected to oil leakage test by the same method as defined in Example 1.

The results of Example 2 and Comparative Examples 2 and 3 are shown in Table 2.

TABLE 2

| | Additives in liquid oil | | | |
|---|---|---|---|---|
| | (soybean oil) | | Content of | |
| | | W/O emulsion | liquid oil | |
| | | (containing | before oil | Oil |
| | | gelatinizable | leakage | leakage |
| | B-100D | substance) | test (%) | rate (%) |
| Example 2 | + | + | 24.0 ± 0.4 | 15.8 ± 2.2* |
| Comparative | − | − | 24.6 ± 0.1 | 57.8 ± 1.8 |
| Example 2 | | | | |
| Comparative | + | − | 24.3 ± 0.2 | 27.1 ± 1.1 |
| Example 3 | | | | |

Note
*As a result of conducting the significant test (student t test) with Comparative Example 3, since p was less than 0.05 (p < 0.05), a significant difference (n = 3) was recognized.

As also apparently recognized from Table 2, the W/O emulsion-containing solid piscicultural diets produced according to the present invention were improved in the effect of preventing leakage of the liquid oil impregnated into the solid material as compared to the current method of Comparative Example 3, and it was therefore confirmed that the W/O emulsion containing the gelatinizable substance exhibited an excellent effect of preventing oil leakage.

(3) Studies on Solid Piscicultural Diets Impregnated with Fish Oil:

In order to prove that the “effect of preventing oil leakage from the solid piscicultural diets by the W/O emulsion” is not a specific effect attained only in the case of using the soybean oil as a vegetable oil used in Examples 1 to 2, the same experiment was conducted using a fish oil as an animal oil.

### Example 3

### Production of Solid Piscicultural Diets Impregnated with W/O Emulsion Using Fish Oil

The same procedure as defined in Example 1 was conducted except that a fish oil (“Kanou” produced by Nikko Yushi Co., Ltd.) was used as the liquid oil in place of the soybean oil to impregnate the liquid oil into the solid piscicultural diets, thereby obtaining an oil-containing solid product. The thus obtained oil-containing solid product was subjected to the same oil leakage test as defined in Example 1.

### Comparative Example 4

The same procedure as defined in Example 1 was conducted except that only the fish oil was used as the liquid oil in place of the W/O emulsion to impregnate the liquid oil into the solid piscicultural diets, thereby obtaining an oil-containing solid product. The thus obtained oil-containing solid product was subjected to the same oil leakage test as defined in Example 1.

### Comparative Example 5

The same procedure as defined in Example 1 was conducted except that a liquid oil prepared by mixing 8.3 parts by weight of decaglycerol behenic acid ester as a polyglycerol fatty acid ester (“B-100D” produced by Mitsubishi-Kagaku Foods Corporation) and 25 parts by weight of a hardened oil (melting point: about 60° C. (broad); “Z-4110” produced by Fuji Oil Co., Ltd.) with 65.7 parts by weight of the fish oil was used in place of the W/O emulsion to impregnate the liquid oil into the solid piscicultural diets, thereby obtaining an oil-containing solid product. The thus obtained oil-containing solid product was subjected to the same oil leakage test as defined in Example 1.

The results of the above Example 3 and Comparative Examples 4 and 5 are shown in Table 3.

TABLE 3

| | Additives in liquid oil | | | |
|---|---|---|---|---|
| | (soybean oil) | | Content of | |
| | | W/O emulsion | liquid oil | |
| | | (containing | before oil | Oil |
| | | gelatinizable | leakage | leakage |
| | B-100D | substance) | test (%) | rate (%) |
| Example 3 | + | + | 23.5 ± 0.6 | 17.6 ± 4.6* |
| Comparative | − | − | 23.9 ± 1.2 | 60.9 ± 1.2 |
| Example 4 | | | | |
| Comparative | + | − | 24.1 ± 0.5 | 33.0 ± 3.0 |
| Example 5 | | | | |

Note
*As a result of conducting the significant test (student t test) relative to Comparative Example 5, since p was less than 0.05 (p < 0.05), a significant difference (n = 3) was recognized.

As also apparently recognized from Table 3, the W/O emulsion-containing solid piscicultural diets produced according to the present invention were improved in the effect of preventing leakage of the liquid oil impregnated into the solid material as compared to those of Comparative Examples, and it was therefore confirmed that the W/O emulsion containing the gelatinizable substance exhibited an excellent effect of preventing oil leakage.

### INDUSTRIAL APPLICABILITY

In accordance with the present invention, it becomes apparent that when the W/O emulsion optionally containing the gelatinizable substance is used as the liquid oil and impregnated into voids of the porous solid material, a larger amount of the liquid oil can be penetrated into the solid material, and the liquid oil can be prevented from being leaked out from the solid material. As a result, various problems concerning oil leakage in porous oil-containing solid products such as solid piscicultural diets can be eliminated, thereby allowing the liquid oil impregnated into the solid material to efficiently exhibit functions thereof.

1. An oil-containing solid product comprising a porous solid material and a W/O emulsion impregnated into pores of the porous solid material.

2. An oil-containing solid product comprising a porous solid material and a W/O emulsion filled in pores of the porous solid material.

3. An oil-containing solid product according to claim 1, wherein a gel polymer is filled in the pores of the porous solid material.

4. An oil-containing solid product according to claim 1, wherein a content of a water phase in the W/O emulsion is 0.01 to 50% by weight.

5. An oil-containing solid product according to claim 1, wherein the water phase in the W/O emulsion contains a water-soluble gelatinizable substance.

6. An oil-containing solid product according to claim 1, wherein the porous solid material is at least one solid material selected from the group consisting of foods, diets, solid fuels, aromatic agents, fertilizers and drugs.

7. A process for producing an oil-containing solid product comprising the step of impregnating a W/O emulsion into a porous solid material.

8. A process according to claim 7, wherein the W/O emulsion contains a water-soluble polymer in a water phase thereof.

9. A process according to claim 8, wherein the water-soluble polymer is gelatinized in pores of the porous solid material.
